(19) United States
(12) Patent Application Publication
(10) Pub. No.: US 2009/0023942 A1
(43) Pub. Date: Jan. 22, 2009

(54) Method for Producing Bi-Functional Ammonium Nitriles

(75) Inventors: Gerd Reinhardt, Kelkheim (DE); Lars Cuypers, Munchen (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT PRODUKTE (DEUTSCHALND) GMBH, Frankfurt am Main (DE)

(21) Appl. No.: 11/885,610

(22) PCT Filed: Feb. 24, 2006
(86) PCT No.: PCT/EP06/01735

§ 371 Date: Jan. 4, 2008

(30) Foreign Application Priority Data
Mar. 1, 2005 (DE) 10 2005 009 137.7

(51) Int. Cl. C07C 253/30; C07C 255/32

(52) U.S. Cl. 558/394

(57) ABSTRACT

The invention relates to a method for producing bifunctional ammonium nitriles by reacting a dihalogenalkyl compound or a dihalogenaryl compound with a dialkylaminoacetonitrile in a polar, aprotic solvent. Bifunctional ammonium nitriles may be used as bleach activators in detergents and cleaners.

The present invention relates to the synthesis of bifunctional ammonium nitrites by a single-stage reaction of dialkylaminoacetonitriles with dihaloalkylene or dihaloarylene.

The patent literature describes ammonium nitrites and their use as bleach activators in detergents and cleaners. By adding these activators, the bleaching effect of aqueous peroxide solutions can be increased to the extent that, at 40° C., essentially the same effect occurs as is otherwise achieved only with the peroxide solution on its own at 95° C. Examples which may be listed are EP-A-303 520, EP-A-458 396, EP-A-464 880 and WO-2003/078 561.

Monofunctional ammonium nitrites of the general formula

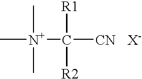

are obtained in EP-A-458 386 in the course of a two-stage reaction. In the first step, the synthesis of a tertiary aminonitrile takes place by reacting the corresponding aldehyde or ketone with potassium cyanide and a secondary amine and, in a second step, the quaternization of the aminonitrile with dimethyl sulfate.

In EP-A-464 880, the quaternization of the aminonitrile takes place with para-methyltoluenesulfonate or para-dodecylalkyl benzenesulfonate.

In the method according to EP-A-303 520, the synthesis of bifunctional ammonium nitrites of the general formula

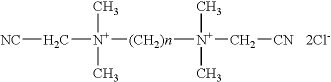

is inter alia described.

In order to arrive at these ammonium nitriles, the corresponding tertiary amine is reacted with chloroacetonitrile to give the respective ammonium nitrile. A problem for the industrial applicability of the method is the high costs of the haloacetonitriles which make the production of this class of substance to be used as bleach activators in detergents and cleaners more expensive.

The object was therefore to find a method for producing bifunctional ammonium nitriles in which the use of haloacetonitrile can be dispensed with.

Surprisingly, it has been found that, by reacting dialkylaminoacetonitrile with dihaloalkyl compounds or haloaryl compounds at 40 to 80° C. in polar-aprotic solvents in a one-pot method, bifunctional ammonium nitriles of this type can be obtained in very pure form and good yields. The use of significantly cheaper raw materials is advantageous.

The present invention thus provides a method for producing bifunctional ammonium nitriles of the formula (1)

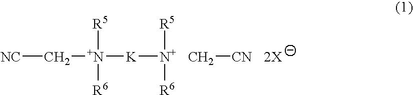

in which K is a group of the formulae

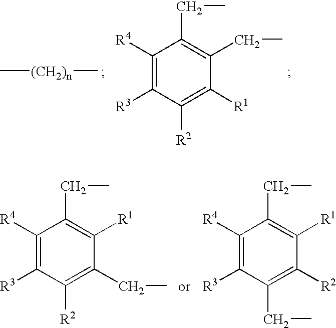

R¹, R², R³ and R⁴, in each case individually, are hydrogen, hydroxyl, C₁- to C₂₄-alkyl, C₂- to C₂₄-alkenyl, C₁-C₄-alkoxy-C₁-C₄-alkyl, phenyl, C₁- to C₄-alkylphenyl, carboxyl, sulfonyl, cyanomethyl, cyano;
R⁵ and R⁶, in each case independently of one another, are a straight-chain or branched-chain C₁- to C₂₄-alkyl, alkenyl or alkyl ether group, and X is an anion, the variable n is an integer from 1 to 16;
where a dialkylaminoacetonitrile of the general formula (2)

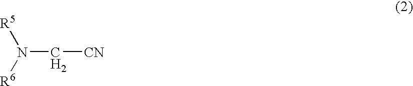

is reacted in a polar, aprotic solvent with a dihaloalkyl compound of the general formula (3)

X—K—X (3)

where R⁵, R⁶, K and X have the abovementioned meanings.

For the method according to the invention, the procedure specifically involves firstly dissolving or suspending the dialkylaminoacetonitrile of the formula (2) in a suitable polar-aprotic solvent. Suitable solvents are, for example: ethyl acetate, n-propyl acetate, isopropyl acetate, n-butyl acetate, isobutyl acetate and mixtures thereof, dimethyl sulfoxide, N-methylpyrrolidone, 1,3-dimethylimidazolidin-2-one. Preference is given to ethyl acetate, dimethylformamide and dimethylacetamide, and particular preference is given to dimethylacetamide since the starting materials have very good solubility in this solvent whereas the end products are sparingly soluble and precipitate out. A dihaloalkyl compound or a haloaryl compound of the general formula (3), without a diluent or as solution, is added dropwise to this solution or suspension. In these dihalo compounds of the formula (3), the two halogen atoms X may be identical or different. The amount of dialkylaminoacetonitrile is 1.8 to 2.5, preferably 2.0 to 2.3, mol equivalents, based on the dihaloalkyl or haloaryl. Suitable dialkylaminoacetonitriles are dimethylaminoacetonitrile, diethylaminoacetonitrile, methylethylaminoacetonitrile, di-n-propylaminoacetonitrile, di-n-butylaminoacetonitrile, di-n-hexylaminoacetonitrile, preferably dimethylaminoacetonitrile and diethylaminoacetonitrile, particularly preferably dimethylaminoacetonitrile. The reaction temperature is generally 20 to 120° C., preferably 30 to 100° C., particularly preferably 40 to 80° C. The reaction proceeds in a period from 1 to 10 hours, preferably 2 to 9 hours, particularly preferably 4 to 8 hours. The resulting product can be separated off from the solvent by filtration, suction filtration, decantation or by centrifugation.

The examples below are intended to illustrate the invention in more detail without limiting it thereto.

### EXAMPLE 1

### Synthesis of α,α′-bis(cyanomethyldimethylammonium)-ortho-xylene dichloride

25 g (0.3 mol) of dimethylaminoacetonitrile were dissolved in 100 ml of N,N-dimethylacetamide. With stirring, a solution of 26 g (0.15 mol) of ortho-xylylene dichloride in 100 ml of N,N-dimethylacetamide was added dropwise at 50° C. The reaction mixture was stirred for 5 hours at 60° C. The reaction mixture was cooled to room temperature and the precipitated solid was filtered off. The filter cake was washed with 50 ml of N,N-dimethyl-acetamide. The solid was dried at 60° C. under reduced pressure. This gave 36.5 g (0.11 mol) of pure α,α′-bis(cyanomethyldimethylammonium)ortho-xylene dichloride, corresponding to a yield of 72%.

m.p.: 165° C.

¹H NMR (D₂O):

### δ=7.83 (4H, m); δ=5.04 (4H, s); δ=4.75 (4H, s); δ=3.28 (12H, s)

### EXAMPLE 2

### Synthesis of α,α′-bis(cyanomethyldimethylammonium)-para-xylene dichloride

25 g (0.3 mol) of dimethylaminoacetonitrile were dissolved in 100 ml of N,N-dimethylacetamide. With stirring, a solution of 26 g (0.15 mol) of para-xylylene dichloride in 100 ml of N,N-dimethylacetamide was added dropwise at 50° C. The reaction mixture was stirred for 5 hours at 60° C. The reaction mixture was cooled to room temperature and the precipitated solid was filtered off. The filter cake was washed with 50 ml of N,N-dimethyl-acetamide. The solid was dried at 60° C. under reduced pressure. This gave 19.2 g (0.06 mol) of pure α,α′-bis(cyanomethyldimethylammonium)para-xylene dichloride, corresponding to a yield of 39%.

m.p.: 208° C. (decomposition)

¹H NMR (D₂O):

### δ=7.78 (4H, m); δ=4.81 (4H, s); δ=4.75 (4H, s); δ=3.33 (12H, s)

### EXAMPLE 3

### Synthesis of N,N,N′,N′-tetramethyl-N,N′-di(cyanomethyl)-1,3-propanediammonium bromide chloride

25 g (0.3 mol) of dimethylaminoacetonitrile were initially introduced into 100 ml of dimethylacetamide and 23.4 g (0.15 mol) of bromochloropropane were added dropwise with stirring at room temperature. The reaction mixture was stirred for 6 hours at 80° C. and then cooled to room temperature. The precipitated solid was filtered off and washed with 3×50 ml of dimethylacetamide. The filter cake was dried under reduced pressure at 60° C. This gave 14.1 g (0.043 mol) of N,N,N′,N′-tetramethyl-N,N′-di(cyanomethyl)-1,3-propanediammonium bromide chloride as white solid, corresponding to a yield of 29%.

m.p.: 178-180° C.

¹H NMR (D₂O):

### δ=4.75 (2H, s); δ=3.75 (4H, t); δ=3.45 (12H, s); δ=2.57-2.51 (2H, m); IR (KBr):

3460 vs, 3410 vs, 3260 m, 3030 s, 2970 s, 2900 vs, 2440 vw, 2275 vw, 1700 vw, 1635 m, 1480 vs, 1450 m, 1425 m, 1380 w, 1340 vw, 1285 w, 1240 vw, 1210 vw, 1195 vw, 1130 vw, 1090 w, 1060 w, 1015 m, 965 s, 940 m, 915 w, 890 s, 790 vw, 755 w

### EXAMPLE 4

### Synthesis of N,N,N′,N′-tetramethyl-N,N′-di(cyanomethyl)-1,5-pentanediammonium dibromide

41.7 g (0.5 mol) of dimethylaminoacetonitrile were initially introduced into 200 ml of N,N-dimethylacetamide, and 57.5 g (0.25 mol) of dibromopentane were added dropwise with stirring at room temperature. The reaction mixture was stirred for 5 hours at 60° C. and then cooled to 5° C. The precipitated solid was filtered off and washed with N,N-dimethylacetamide. The filter cake was dried at 60° C. under reduced pressure. This gave 30.4 g (0.076 mol) of N,N,N′,N′-tetramethyl-N,N′-di(cyanomethyl)-1,5-pentanediammonium dibromide as white solid, corresponding to a yield of 30.5%.

m.p.: 175° C. (decomposition)

¹H NMR (D₂O):

### δ=4.75 (2H, s); δ=3.62 (4H, t); δ=3.36 (12H, s); δ=1.99-19.5 (4H, m); δ=1.55-1.52 (2H, m)

### IR (KBr):

3035 m, 3010 m, 2945 s, 2900 vs, 2740 w, 2440 vw, 2240 vw, 1785 vw, 1620 vw, 1470 s, 1450 s, 1420 w, 1405 m, 1370 w, 1340 w, 1295 vw, 1280 vw, 1225 vw, 1210 w, 1165 w, 1095 w, 1050 w, 1015 w, 1005 w, 990 w, 970 m, 960 m, 930 m, 890 m, 840 vw, 770 vw, 750 vw, 730 vw

### EXAMPLE 5

### Bleaching Power of Bifunctional Ammonium Nitriles

The bleaching power of the cyanomethylammonium salts was investigated in a Linitest device (Heraus) at 20° C., 40° C. and 60° C. For this purpose, 2 g/l of a bleach-free basic detergent (WMP, WFK, Krefeld) and 1 g/l of sodium perborate monohydrate (Degussa) were dissolved in water of hardness grade 3.

200 mg/l of activator were then added. The washing time was 30 min. The sections of fabric were then rinsed with water, dried and ironed. The bleach test fabrics used were tea BC-1, curry BC-4 and grass CS-8 (WFK Testgewebe GmbH, Krefeld) on cotton. As the bleaching result, the difference in reflectance, measured using an Elrepho device, after washing compared to the fabric washed with 2 g/l of WMP and 1 g/l of sodium perborate monohydrate was evaluated.

ΔΔR=ΔR(formulation+persalt+activator)−ΔR(formulation*persalt)

Bleach compositions containing the cationic nitrile compounds according to the invention of examples 1 and 2 according to the invention and also comparison substances 3 and 4 were prepared and tested.

Compounds 1 to 4 have the following structure:

TABLE 1
Test results (ΔΔR values) for activators 1 to 4

| | |
|---|---|
| | 1 |
| 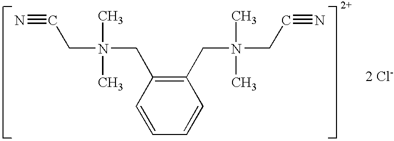 | |
| | 2 |
| 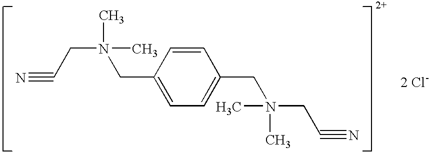 | |
| | 3 |
| 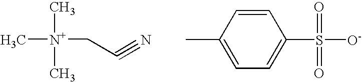 | |
| | 4 |
| 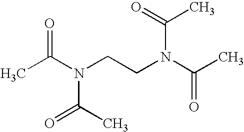 | |

| | | Acti- | Acti- | Acti- | Acti- |
|---|---|---|---|---|---|
| Soiling | T [° C.] | vator 1 | vator 2 | vator 3 | vator 4 |
| Tea BC-1 | 20 | 11.1 | n.d. | 4.4 | 0.9 |
| Tea BC | 40 | 11.6 | n.d. | 4.6 | 2.1 |
| Tea BC | 60 | 10.5 | n.d. | 3.1 | 2.7 |
| Curry BC-4 | 20 | 4.1 | 3.7 | n.d. | n.d. |
| Curry BC-4 | 40 | 3.9 | 4.1 | n.d. | n.d. |
| Grass CS-8 | 20 | 11.8 | n.d. | 4.1 | 2.0 |
| Grass CS | 40 | 11.2 | n.d. | 6.2 | 4.9 |
| Grass CS | 60 | 4.0 | n.d. | 4.0 | 1.4 |

n.d.: not determined

The investigations show that the cationic nitrites according to the invention develop a better bleaching effect on all soilings than the activators of the prior art.

Further useful properties of the cationic nitrites are low color damage and low fiber damage.

1. A method for producing bifunctional ammonium nitriles of the formula (1) 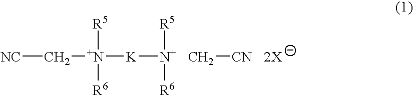
in which K is a group of the formulae 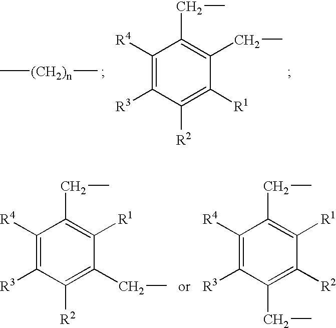
R¹, R², R³ and R⁴, in each case individually, are hydrogen, hydroxyl, C₁- to C₂₄-alkyl, C₂- to C₂₄-alkenyl, C₁-C₄-alkoxy-C₁-C₄-alkyl, phenyl, C₁- to C₄-alkylphenyl, carboxyl, sulfonyl, cyanomethyl, cyano;
R⁵ and R⁶, in each case independently of one another, are a straight-chain or branched-chain C₁- to C₂₄-alkyl, alkenyl or alkyl ether group, and
X is an anion,
the variable n is an integer from 1 to 16;
said process comprising reacting a dialkylaminoacetonitrile of the general formula (2) 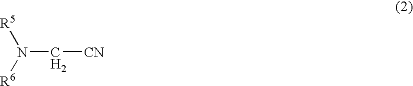
in a polar, aprotic solvent with a dihaloalkyl compound of the general formula (3)
X—K—X (3)
where R⁵, R⁶, K and X have the abovementioned meanings.

2. The method as claimed in claim 1, wherein the dialkylaminoacenitrile is selected from the group consisting of dimethylamino-acetonitrile, diethylaminoacetonitrile, methylethylaminoacetonitrile, di-n-propylaminoacetonitrile, di-n-butylaminoacetonitrile and di-n-hexylamino-acetonitrile.

3. The method as claimed in claim 1, wherein the dialkylaminoacenitrile is selected from the group consisting of dimethylamino-acetonitrile and diethylaminoacetonitrile.

4. The method as claimed in claim 1, wherein the dialkylaminoacenitrile is dimethylaminoacetonitrile.

5. The method as claimed in claim 1, wherein the dialkylaminoacetonitrile is present in the amount of 1.8 to 2.5 mol equivalents based on the compound of the formula (3).

6. The method as claimed in claim 1, wherein the dialkylaminoacetonitrile is present in the amount of 2.0 to 2.3 mol equivalents based on the compound of the formula (3).

7. The method as claimed in claim 1, wherein said polar, aprotic solvent is selected from the group consisting of ethyl acetate, n-propyl acetate, isopropyl acetate, n-butyl acetate, isobutyl acetate, dimethyl sulfoxide, N-methylpyrrolidone, 1,3-dimethylimidazolidin-2-one and mixtures thereof.

8. The method as claimed in claim 1, wherein said polar, aprotic solvent is selected from the group consisting of ethyl acetate, dimethylformamide and dimethylacetamide.

9. The method as claimed in claim 1, wherein said polar, aprotic solvent is dimethylacetamide.

10. The method as claimed in claim 1, wherein said reacting is carried out at a temperature of 20 to 120° C.

11. The method as claimed in claim 1, wherein said reacting is carried out at a temperature of 30 to 100° C.

12. The method as claimed in claim 1, wherein said reacting is carried out at a temperature of 40 to 80° C.

13. The method as claimed in claim 1, wherein said reacting includes a reaction time of 1 to 10 hours.

14. The method as claimed in claim 1, wherein said reacting includes a reaction time of 2 to 9 hours.

15. The method of claim 1, wherein said reacting includes a reaction time of 4 to 8 hours.

16. A detergent, cleaner or disinfectant comprising a bifunctional ammonium nitrile of the general formula (1) 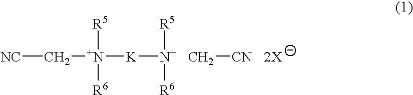
in which K is a group of the formulae 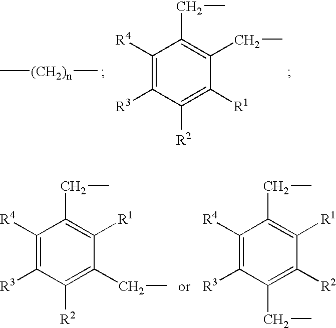
R¹, R², R³ and R⁴, in each case individually, are hydrogen, hydroxyl, C₁- to C₂₄-alkyl, C₂- to C₂₄-alkenyl, C₁-C₄-alkoxy-C₁-C₄-alkyl, phenyl, C₁- to C₄-alkylphenyl, carboxyl, sulfonyl, cyanomethyl, cyano;
R⁵ and R⁶, in each case independently of one another, are a straight-chain or branched-chain C₁- to C₂₄-alkyl, alkenyl or alkyl ether group, and
X is an anion,
the variable n is an integer from 1 to 16;
produced as claimed in claim 1.

17. A machine dishwashing composition comprising a bifunctional ammonium nitrile of the general formula (1) 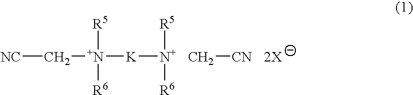
in which K is a group of the formulae 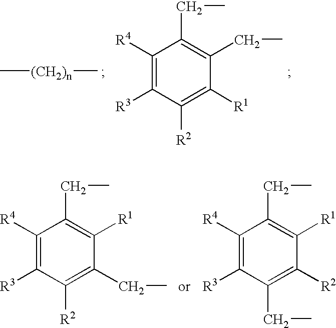
R¹, R², R³ and R⁴, in each case individually, are hydrogen, hydroxyl, C₁- to C₂₄-alkyl, C₂- to C₂₄-alkenyl, C₁-C₄-alkoxy-C₁-C₄-alkyl, phenyl, C₁- to C₄-alkylphenyl, carboxyl, sulfonyl, cyanomethyl, cyano;
R⁵ and R⁶, in each case independently of one another, are a straight-chain or branched-chain C₁- to C₂₄-alkyl, alkenyl or alkyl ether group, and
X is an anion,
the variable n is an integer from 1 to 16;
produced as claimed in claim 1.

18. A detergent formulation comprising a bifunctional ammonium nitrile of the general formula (1) 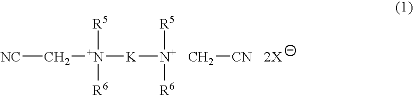
in which K is a group of the formulae 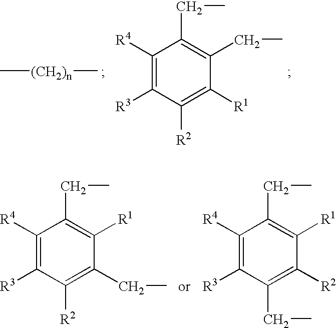
R¹, R², R³ and R⁴, in each case individually, are hydrogen, hydroxyl, C₁- to C₂₄-alkyl, C₂- to C₂₄-alkenyl, C₁-C₄-alkoxy-C₁-C₄-alkyl, phenyl, C₁- to C₄-alkylphenyl, carboxyl, sulfonyl, cyanomethyl, cyano;
R⁵ and R⁶, in each case independently of one another, are a straight-chain or branched-chain C₁- to C₂₄-alkyl, alkenyl or alkyl ether group, and
X is an anion,
the variable n is an integer from 1 to 16;
produced as claimed in claim 1.
